(19) United States
(12) Patent Application Publication
(10) Pub. No.: US 2008/0292609 A1
(43) Pub. Date: Nov. 27, 2008

(54) Use of Arginase in Combination with 5FU and Other Compounds for Treatment of Human Malignancies

(76) Inventors: Ning Man Cheng, Hong Kong (CN); Yun Chung Leung, Hong Kong (CN); Wai Hung Lo, Hong Kong (CN)

Correspondence Address: EAGLE IP LIMITED, 22/F., KWAI HUNG HOLDINGS CENTRE, 89 KING'S ROAD, NORTH POINT, HK

(21) Appl. No.: 11/751,050

(22) Filed: May 21, 2007

(51) Int. Cl. A61K 38/46; A61P 35/00

(52) U.S. Cl. 424/94.6

(57) ABSTRACT

The present invention provides a method for treating cancer in a human patient by reducing the physiological arginine levels to below 10 μM in combination with administering an anti-neoplastic compound. The present invention also provides a pharmaceutical composition comprising an arginine reducing compound, such as pegylated human arginase I, and an anti-neoplastic compound, such as 5-fluorouracil, for the treatment of malignancies in a human patient.

This application claims the benefit of U.S. Provisional Application Ser. No. U.S. 60/633,398, filed on 3 Dec. 2004 which is incorporated by reference herein in its entirely.

### FIELD OF INVENTION

The present invention is related to pharmaceutical compositions for the treatment of human malignancies containing arginase.

### BACKGROUND OF INVENTION

Cancer remains one of the most difficult to treat human diseases. For certain forms of cancer, such as liver cancer, there is no known effective drug.

Arginine degrading enzymes can be developed as drugs to treat cancer. Arginase I (EC 3.5.3.1; L-arginine amidinohydrolase), is a key mammalian liver enzyme that catalyzes the final step in urea formation in the Urea cycle, converting arginine into ornithine and urea. PCT publication WO 2004/000349 discloses a pharmaceutical composition containing human recombinant arginase. U.S. patent application Ser. No. 10/757,843 discloses a different arginine degrading enzyme, arginine deiminase modified with polyethylene glycol, used for the treating of cancer, and the treating and/or inhibiting of metastasis. Lastly, U.S. patent application Ser. No. 09/905,201 discloses a therapeutic composition and method for treatment of cancer comprising arginine decarboxylase of E. coli and modifications thereof.

It is an object of the present invention to provide improved methods of treatment and compositions for the treatment of cancer.

### SUMMARY OF INVENTION

Accordingly, one aspect of the present invention teaches a kit comprising at least one therapeutic dose of an arginine reducing compound and at least one therapeutic dose of an anti-neoplastic compound such as 5-fluorouracil (5FU), for the treatment of human malignancies. The arginine reducing compound is an enzyme or a compound that is capable of degrading or removing arginine from the subject, so as to achieve a physiological arginine level of below 10 μM. Some examples of arginine reducing compounds include, but are not limited to, arginine degrading enzymes such as arginase, human arginase I, arginine deiminase and arginine decarboxylase, modifications thereof or combinations thereof. An anti-neoplastic compound may be, but is not limited to, an alkylating agent, antimetabolite agent, antimitotic agent, DNA production inhibiting agent or DNA repair inhibiting agent, etc.

According to another aspect of the present invention there is provided a method for treating cancer in a human patient, the method comprising reducing the physiological arginine levels in the patient to below 10 μM combined with a suitable anti-neoplastic or antimetabolite compound such as 5FU. The reduction of physiological arginine levels in a human patient may be achieved through various treatments which include, but are not limited to, embolization, dialysis or administration of an arginine reducing compound.

According to a further aspect of the present invention there is provided a pharmaceutical composition comprising an arginine reducing compound and an anti-neoplastic compound.

According to yet another aspect of the present invention there is provided a use of an arginine reducing compound in combination with an anti-neoplastic compound for the manufacture of a medicament for the treatment of cancer. The arginine reducing compound may be an arginine degrading enzyme. Some examples of such arginine degrading enyzmes include, but are not limited to, arginase, arginine deiminase, arginine decarboxylase, or modifications and combinations thereof. The arginase may also be human arginase I or pegylated human arginase. The anti-neoplastic compound may, for example, be an alkylating agent, antimetabolic agent, antibiotic agent, DNA production inhibiting agent or DNA repair inhibiting agent, etc. The anti-neoplastic compound may preferably be 5-fluorouracil.

### BRIEF DESCRIPTION OF FIGURES

FIG. 1 shows plasmid map of pAB101. This plasmid carries the gene encoding Arginase (arg) and only replicates in E. coli but not in B. subtilis.

FIGS. 2A, 2B and 2C show nucleotide sequence and its deduced amino acid sequence of the human Arginase I. FIG. 2A shows the nucleotide sequence (SEQ ID NO: 1) from EcoRI/MunI to XbaI sites of plasmid pAB101. Nucleotide (nt) 1-6, EcoRI/MunI site; nt 481-486, -35 region of promoter 1; nt 504-509, -10 region of promoter 1; nt 544-549, -35 region of promoter 2; nt 566-571, -10 region of promoter 2; nt 600-605, ribosome binding site; nt 614-616, start codon; nt 632-637, NdeI site; nt 1601-1603, stop codon; nt 1997-2002, XbaI site.

FIG. 2B shows the encoding nucleotide sequence (SEQ ID NO: 2) and its corresponding encoded amino acid sequence (SEQ ID NO: 3) of a modified human Arginase. Nucleotide 614-1603 from FIG. 2A is an encoding region for the amino acid sequence of the modified Arginase. The 6xHis (SEQ ID NO: 4) tag at the N-terminus is underlined. Translation stop codon is indicated by asterisk.

FIG. 2C shows the encoding nucleotide sequence (SEQ ID NO: 8) and its corresponding encoded amino acid sequence (SEQ ID NO: 9) of the normal human Arginase I.

FIG. 3 is a schematic drawing of the construction of a B. subtilis prophage allowing expression of Arginase.

FIG. 4 shows the comparison of average tumor size for four groups of nude mice which have tumors induced by implantation with tumor cells. The four groups are negative control, arginase (BCT) alone, arginase (BCT) and arginase (BCT) in combination with 5FU, an anti-neoplastic and antimetabolite compound.

### DETAILED DESCRIPTION OF THE PREFERRED EMBODIMENTS

As used herein and in the claims, “comprising” means including the elements that follow but not excluding others.

“Combined administration” or “combined with administering” merely refers to a general period of time in which both arginase and an anti-neoplastic agent are administered to the human body for the treatment of human malignancies. It does not restrict the method of treatment to a simultaneous administration of the two types of compounds. When in reference to treatments that do not require the administration of a compound (such as dialysis or embolization), “combined with” merely refers to a general period of time in which the two steps of treatment for cancer are performed, this includes and is not limited to the possibility of simultaneous performance of the two steps.

Similarly, the term “medicament” may refer to two different compounds applied at different times, as long as the two compounds belong to the same combination treatment.

As used herein, the term “pegylated Arginase” refers to Arginase of present invention modified by pegylation to increase the stability of the enzyme and minimize immunoreactivity. In particular, arginase I, in both its modified and unmodified forms is the preferred arginase. Pegylated arginase I may also be referred to as “BCT” and is used interchangeably in this application.

Human arginase I and other amino acid sequences as used herein include amino acid sequences that are substantially the same, meaning that they may have “slight and non-consequential sequence variations” from the actual sequences disclosed herein. Species with sequences that are substantially the same are considered to be equivalent to the disclosed sequences and as such are within the scope of the appended claims. In this regard, “slight and non-consequential sequence variations” means that the amino acid sequences are functionally equivalent to the sequences disclosed and/or claimed herein. Functionally equivalent sequences will function in substantially the same manner to produce substantially the same compositions as the amino acid compositions disclosed and claimed herein.

As used herein, the term “half-life” (½-life) refers to the time that would be required for the concentration of the Arginase in human plasma in vitro, to fall by half.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference to describe and disclose specific information for which the reference was cited in connection with.

Arginase may be obtained from Ikemoto et al. (Ikemoto et al. Biochem J. 1990. 270: 697-703) or by the method disclosed in PCT publication WO 2004/000349. The arginase may also be produced using the methods described below.

All references cited above and in the following description are incorporated by reference herein. The practice of the invention is exemplified in the following non-limiting examples. The scope of the invention is defined solely by the appended claims, which are in no way limited by the content or scope of the examples. Examples 8 and 9 described below are alternative ways of practicing the present invention.

### EXAMPLE 1

### Construction of the Recombinant Strain LLC101

### (a) Isolation of the Gene Encoding Human Arginase I

The gene sequence of human Arginase I was published in 1987 (Haraguchi Y. Proc Natl Acad Sci. 1987. 84: 412-415) and primers designed therefrom. Polymerase chain reaction (PCR) was performed to isolate the gene encoding a human Arginase using the Expand High Fidelity PCR System Kit (Roche, Indianapolis, USA). Primers Arg1 (5′-CCAAACCATATGAGCGCCAAGTCCAGAACCATA-3′) (SEQ ID NO: 5) and Arg2 (5′-CCAAACTCTAGAATCACATTTTTTGAATGACATGGACAC-3′) (SEQ ID NO: 6), respectively, were purchased from Genset Singapore Biotechnology Pte Ltd. Both primers have the same melting temperature (Tm) of 72 degree C. Primer Arg1 contains a NdeI restriction enzyme recognition site (underlined) and primer Arg2 contains a XbaI site (underlined). These two primers (final concentration 300 nM of each) were added to 5 μl of the human liver 5′-stretch plus cDNA library (Clontech, California, USA) in a 0.2-ml micro-tube. DNA polymerase (2.6 units, 0.75 μl), the four deoxyribonucleotides (4 μl of each; final concentration 200 μM of each) and reaction buffer (5 μl) and dH₂O (17.75 μl) were also added. PCR was performed using the following conditions: pre-PCR (94 degree C., 5 min), 25 PCR cycles (94 degree C., 1 min; 57 degree C., 1 min; 72 degree C., 1 min), post-PCR (72 degree C., 7 min). PCR product (5 μl) was analyzed on a 0.8% agarose gel and a single band of 1.4 kb was observed. This DNA fragment contains the gene encoding Arginase.

(b) Isolation of Plasmid pSG1113

Plasmid pSG1113, which is a derivative of plasmid pSG703 (Thornewell S J et al. Gene. 1993. 133: 47-53), was isolated from the E. coli DH5α clone carrying pSG1113 by using the Wizard Plus Minipreps DNA Purification System (Promega, Wisconsin, USA) following the manufacturer's instruction. This plasmid, which only replicates in E. coli but not in B. subtilis, was used as the vector for the sub-cloning of the Arginase gene.

(c) Sub-Cloning the 1.4 kb PCR Product Into Plasmid pSG1113 to Form Plasmid pAB101

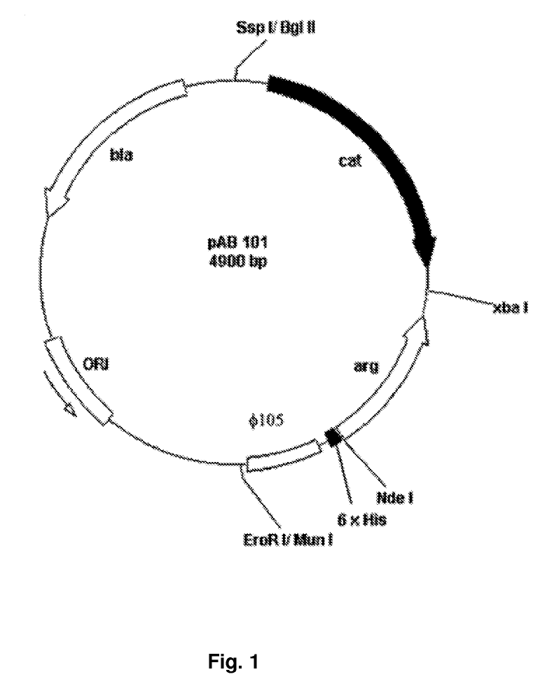

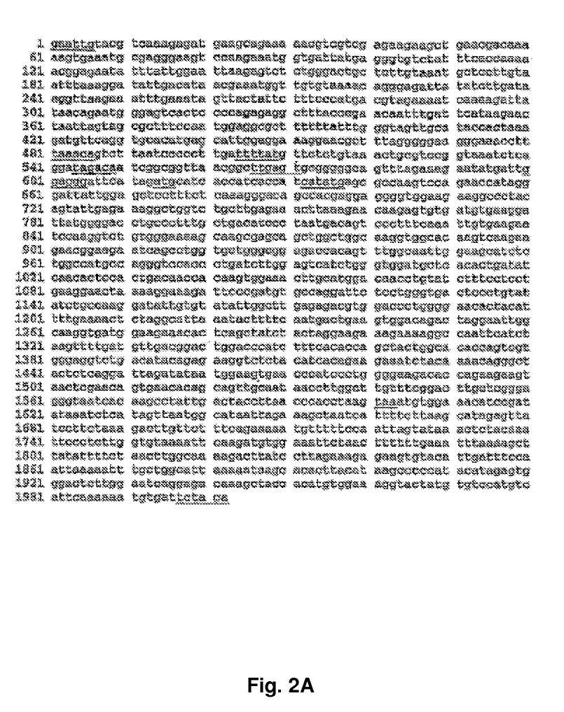

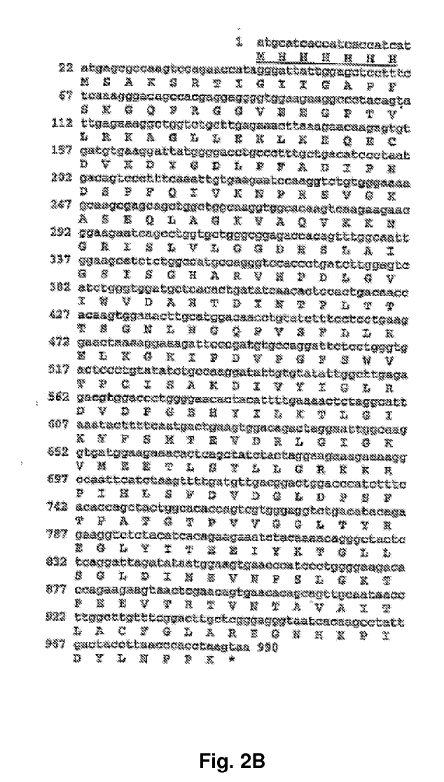

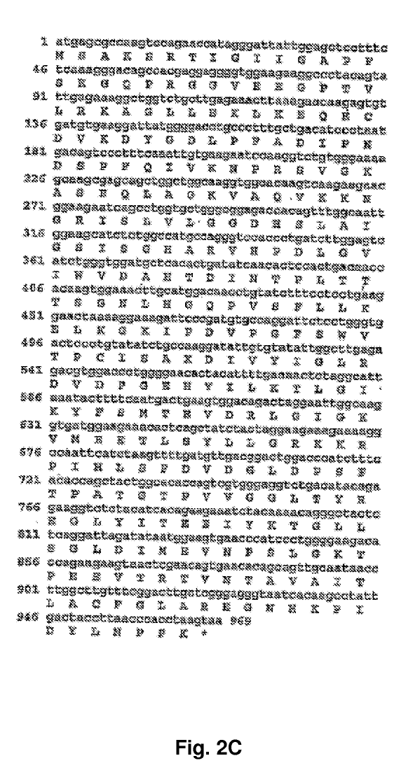

The PCR product, prepared using the above protocol, was treated with restriction endonucleases NdeI and XbaI (Promega, Wisconsin, USA) in a reaction medium composed of 6 mM Tris-HCl (pH 7.9), 6 mM MgCl₂, 150 mM NaCl, 1 mM DTT at 37 degree C. for 1.5 h. After completion of the treatment, the reaction mixture was subjected to agarose gel (0.8%) electrophoresis, and the 1.4 kb DNA fragment was recovered from the gel by using the Qiaex II Gel Extraction Kit (Qiagen, California, USA). Separately, the plasmid pSG1113 was treated with the same restriction endonucleases in the same way. After completion of the treatment, the reaction mixture was subjected to agarose gel (0.8%) electrophoresis, and a DNA fragment having a size of about 3.5 kb was recovered from the gel. This DNA fragment was joined by using T4 DNA ligase to the above 1.4 kb DNA fragment. The ligation mixture was used to transform E. coli XLI-Blue using the conventional calcium method (Sambrook J et al. Molecular Cloning, A Laboratory Manual, second edition. Cold Spring Harbor Laboratory Press, New York. 1989) and plated on nutrient agar plate containing 100 μg/ml ampicillin. Colonies were screened for a plasmid with the appropriate insert by restriction analysis. The plasmid constructed was designated pAB101 (FIG. 1). ORI is the E. coli origin of replication and bla is the ampicillin resistant marker gene. DNA sequencing was performed with primers Arg1 (SEQ ID NO: 5), Arg2 (SEQ ID NO: 6) and Arg6 (5′-CTCTGGCCATGCCAGGGTCCACCC-3′) (SEQ ID NO: 7) to confirm the identity of the gene encoding Arginase (FIG. 2A, B, C).

(d) Construction of the Novel Recombinant B. subtilis Prophage Strain LLC101

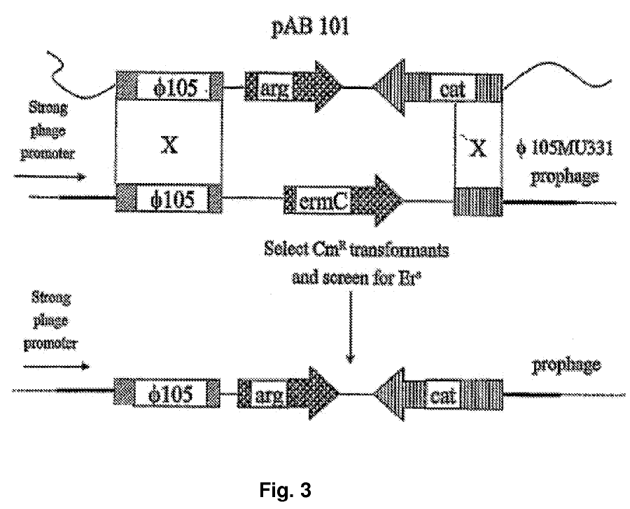

The plasmid pAB101 was extracted and purified from the clone carrying the pAB101 by using the Wizard Plus Minipreps DNA Purification System (Promega, Wisconsin, USA). In the plasmid pAB101 (FIG. 1), the Arginase gene (arg) was flanked by the 0.6 kb MunI-NdeI φ105 phage DNA fragment (labeled as “φ105”) and the cat gene (FIG. 1 and FIG. 3). This plasmid DNA (1 g) was used to transform competent B. subtilis 1A304(φ105MU331) according to the known method (Anagnostopoulos C and Spizizen J. J Bacteriol. 1961. 81: 741-746). The B. subtilis strain 1A304 (φ105MU331) was obtained from J. Errington (Thornewell S et al. 1993. Gene. 133: 47-53). The strain was produced according to the publications by Thornewell, S. et al., 1993, Gene 133, 47-53 and by Baillie, L. W. J. et al., 1998, FEMS Microbiol. Letters 163, 43-47, which are incorporated herein in their entirety. Plasmid pAB101 (shown linearized in FIG. 3) was transformed into the B. subtilis strain 1A304 (φ105MU331) with selection for the Cm^{R} marker, and the transformants were screened for an Er^{S} phenotype. Such transformants should have arisen from a double-crossover event, as shown in FIG. 3, placing transcription of the Arginase gene (arg) under the control of the strong phage promoter (Leung and Erington. Gene. 1995. 154: 1-6). The thick lines represent the prophage genome, broken lines the B. subtilis chromosome, and thin lines plasmid DNA. The genes are shown in FIG. 3 as shaded arrows pointing in the direction of transcription and translation. Regions of homology are bounded by broken vertical lines and homologous recombination events by ‘X’.

Fifty-two chloramphenicol resistant (Cm^{R}) colonies were obtained from plating 600 μl of the transformed cells on an agar plate containing chloramphenicol (5 g/ml). Ten of these colonies were selected randomly and streaked onto an agar plate containing erythromycin (20 μg/ml) and one of these colonies did not grow, indicating that it was erythromycin sensitive (Er^{S}). This chloramphenicol resistant but erythromycin sensitive colony was thus isolated and named as LLC101. In the chromosome of this newly constructed prophage strain, the erythromycin resistance gene (ermC) was replaced by the Arginase gene (arg) by a double crossover event in a process of homologous recombination. The 0.6 kb MunI-NdeI φ105 phage DNA fragment (labeled as “φ105”) and the cat gene provided the homologous sequences for the recombination. In this way, the Arginase gene was targeted to the expression site in the prophage DNA of B. subtilis 1A304 (φ105MU331) and the Arginase gene was put under the control of the strong thermoinducible promoter (Leung Y C and Errington J. Gene. 1995. 154: 1-6).

### EXAMPLE 2

### Fermentation of B. Subtilis LLC101 Cells

The fed-batch fermentation was carried out in a 2-liter fermentor at 37 degree C., pH 7.0 and dissolved oxygen 20% air saturation. The feeding medium contained 200 g/L glucose, 2.5 g/L MgSO₄.7H₂O, 50 g/L tryptone, 7.5 g/L K₂HPO₄ and 3.75 g/L KH₂PO₄. The medium feeding rate was controlled with the pH-stat control strategy. In this strategy, the feeding rate was adjusted to compensate the pH increase caused by glucose depletion. This control strategy was first implemented when the glucose concentration decreased to a very low level at about 4.5-h fermentation time. If pH>7.1, 4 mL of feeding medium was introduced into the fermentor. Immediately after the addition of glucose, the pH value would decrease below 7.1 rapidly. After approximate 10 min, when the glucose added was completely consumed by the bacterial cells, the pH value would increase to a value greater than 7.1, indicating that another 4 mL of feeding medium was due to be added into the fermentor. Heat shock was performed at 5-6 h when the culture density (OD_{600 nm}) was between 12.0 and 13.0. During the heat shock, the temperature of the fermentor was increased from 37 degree C. to 50 degree C. and then cooled immediately to 37 degree C. The complete heating and cooling cycle took about 0.5 h. Cells were harvested for separation and purification of Arginase at 3 h and 6 h after heat shock. In this example, the aforementioned strain produced active human Arginase in an amount of at least about 162 mg per L of the fermentation medium at 6 h after heat shock.

### EXAMPLE 3

### Purification of Arginase at 6 H After Heat Shock After Fed-Batch Fermentation at Low Cell Density

Fed-batch fermentation was performed as described in Example 2. The cell culture (650 ml) collected at 6 h after heat shock at OD 12.8 was centrifuged at 5,000 rpm for 20 min at 4 degree C. to pellet the cells. The wet weight of the cells was 24 g. The culture supernatant liquor was discarded and the cell pellet was stored at −80° C. The cells are stable at this temperature for a few days. To extract intracellular proteins, the cell pellet was resuspended in 140 ml solubilization buffer [50 mM Tris-HCl (pH 7.4), 0.1 M NaCl, 5 mM MnSO₄, lysozyme (75 μg/ml)]. After incubation at 30 degree C. for 15 min, the mixture was sonicated for eight times, each time lasted for 10 s (the total time was 80 s), at 2 min intervals using the Soniprep 150 Apparatus (MSE, Osaka, Japan). About 500 units of deoxyribonuclease I (Catalog No. D 4527, Sigma, Missouri, USA) was added and the mixture was incubated at 37 degree C. for 10 min to digest the chromosomal DNA. After centrifugation at 10,000 rpm for 20 min at 4 degree C., the supernatant, containing the crude protein extract, was assayed for the presence of the Arginase activity and analyzed by SDS-PAGE (Laemmli. Nature. 1970. 227: 680-685).

A 5-ml HiTrap Chelating column (Pharmacia, New Jersey, USA) was equilibrated with 0.1 M NiCl₂ in dH₂O, for 5 column volumes. The crude protein extract (140 ml) was loaded onto the column. Elution was performed with a linear gradient (0-100%) at a flow rate of 5 m/min for 15 column volumes under the following conditions: Buffer A=start buffer [0.02 M sodium phosphate buffer (pH 7.4), 0.5 M NaCl]; Buffer B=start buffer containing 0.5 M imidazole. Fractions with arginase activity and high arginase purity (fractions13-24) were pooled (24 ml) and diluted ten times with start buffer [0.02 M sodium phosphate buffer (pH 7.4), 0.5 M NaCl]. This was loaded onto a second 5-ml HiTrap Chelating column (Pharmacia, New Jersey, USA), repeating the same procedure as above. Fractions 12-24 containing high protein levels as measured by protein gel were pooled as the arginase fractions and salt was removed using a 50-ml HiPrep 26/10 desalting column (Pharmacia, New Jersey, USA) with the following conditions: flow rate=10 ml/min, buffer=10 mM Tris-HCl (pH 7.4) and length of elution=1.5 column volume. The protein concentration was measured by the method of Bradford (Bradford M. Anal Biochem. 1976. 72: 248-254).

In this example, a total of 85.73 mg of Arginase was purified from 650 ml cell culture. The yield of purified Arginase was estimated to be 132 mg/l cell culture or 3.57 mg/g wet cell weight.

### EXAMPLE 4

### Preparation of Highly Active Pegylated Arginase

The purified Arginase as prepared using the methods described above was used for pegylation. Using the previous examples to illustrate the method, an arginase sample with specific activity=518 I.U./mg was dissolved in PBS buffer before carrying out pegylation.

The mPEG-SPA (cat. no. 2M4MOH01, Nektar, USA), MW 5,000 (5.82 g) was added into 555 ml of the purified Arginase (813.64 mg, 1.466 mg/ml) solution slowly in a 1 L beaker and then stirred for 2 h 40 min at room temperature (mole ratio of Arginase:mPEG-SPA=1:50). The mixture was then dialyzed extensively by ultra-dialysis against 15 L of PBS buffer using the F50(S) capillary dialyzer (Fresenius Medical Care, Bad Homburg, Germany) to remove all the unincorporated PEG. The mPEG-SPA uses amino groups of lysines and the N-terminus of the protein as the site of modification. In this example, the measured specific activity of the pegylated Arginase was as high as 592 I.U./mg.

### EXAMPLE 5

### ½-Life Determination of Pegylated Arginase in Vitro Using the Method in Human Blood Plasma

Purified Arginase (1 mg) was dissolved in 1 ml of 125 mM borate buffer solution (pH 8.3) on ice. Activated PEG (mPEG-SPA, MW 5,000) (7.14 mg) was added into the protein solution slowly at a mole ratio of Arginase:PEG=1:50. The mixture was stirred on ice for 2.5 h.

Pegylated Arginase (305.6 μl) at a concentration of 1 mg/ml was added into human plasma (1 ml) and the final concentration of pegylated Arginase was 0.24 mg/ml. The mixture was divided into 20 aliquots in eppendorf tubes (65 μl mixture in each eppendorf tube) and then incubated at 37° C. A 1-2 μl portion of the mixture from each eppendorf tube was used to test the Arginase activity. In this example, the ½-life was determined to be approximately 3 days. It took about 3 days to reduce the relative activity from 100% to 50%.

### EXAMPLE 6

### The Response of Tumor Size to Combinatorial Administration of Arginase (BCT) and 5FU in Mice

In this example, arginase (BCT) was used in combination with 5FU. Hep 3B cells obtained from the American Type Culture Collection (ATCC) were propagated through four passages according to the supplier's recommendations before 40 nude mice were implanted with a tumor of this cell line of at least 3 mm³.

When the tumors reached an average diameter of 5 mm, the mice were divided randomly into four groups of 10 animals each. These are: Group 1, negative control (0.2 ml of 0.9% normal saline as negative control); Group 2, 250 IU of BCT; Group 3, combination of 250 IU arginase (BCT) and 10 mg/kg 5FU (5-fluorouracil) (Ebewe Arzneimittel Ges.m.b.H., Austria, Europe); and Group 4, 10 mg/kg 5FU. The animals were treated by intraperitoneal injection of the compound(s) or normal saline once a week.

The implanted animals were observed once every two days for growth of the solid tumor in situ by digital caliper measurements to tumor size and weight. The tumor size was the average of two perpendicular diameters and one diagonal diameter. The tumor weight was taken to be the (length×width²)/2; assuming a specific gravity of 1.0 g/cm³.

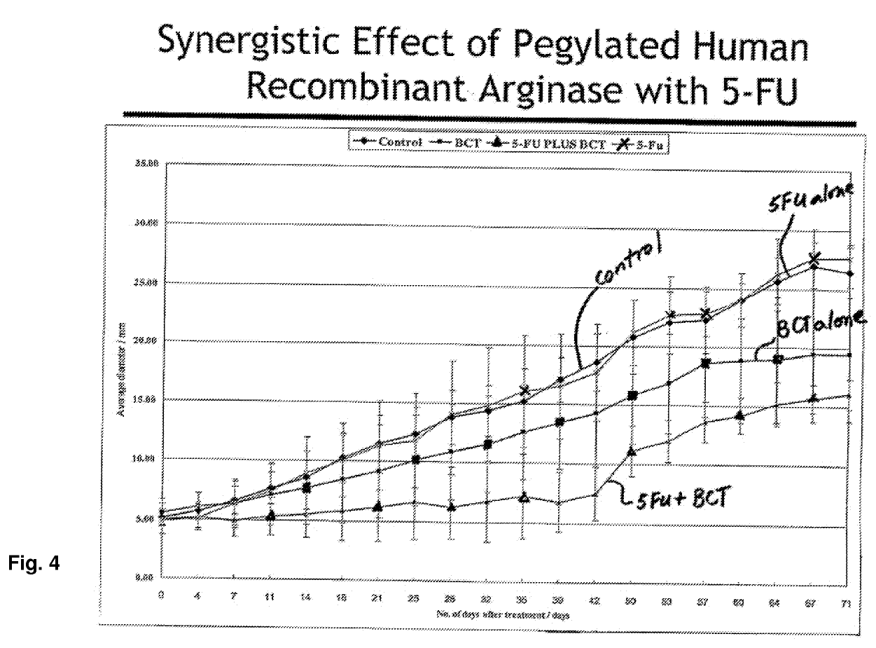

The tumour growth was measured for 71 days and is shown in FIG. 4. Statistical analysis was performed by SPSS 11.0 software (SPSS, Chicago, USA), based on Kaplan-Meier estimation and groups were compared by the log-rank test.

It may be seen from FIG. 4 that the combinatorial administration of arginase (BCT) and 5FU yields unexpected synergistic results when compared to either compound on its own. Furthermore, the doses of arginase (BCT) used were lower than what is required to see efficacy when BCT is used alone.

### EXAMPLE 7

### Treatment Protocol of Patients Using Exogenously Administered Arginase (BCT)

When BCT is administered into a patient, the blood samples of the patients may be taken daily throughout treatment for arginine levels, Arginase (BCT) activities, complete blood picture and full clotting profile. Renal and liver functions are taken at least every other day, or sooner if deemed necessary.

Vital signs (BP, Pulse, Respiratory rate, Oximeter reading) are taken every 15 minutes for 1 hour after commencement of Arginase infusion then hourly until stable. Thereafter, vital signs are taken at the discretion of the treating physician.

On day 1 BCT is infused over 30 minutes at 2,000 IU per kg. Thereafter, BCT is infused weekly for 8-12 weeks. This may be continued if anti-tumour activity is observed. Twenty minutes before each BCT infusion, pre-medication with dipheneramine 10 mg iv. and hydrocortisone 100 mg iv. is given.

As for 5FU, it may administered at 125 mg per meter square by short infusion every day from day 1 to day 5. Each 5FU infusion from day 1 to day 5 is preceded by administration of Folinic acid of 50 mg per meter square. This same 5FU and Folinic acid treatment may be repeated every 4 weeks.

### EXAMPLE 8

### Using Embolization as a Method to Reduce Physiological Arginine Levels in Combination with an Anti-Neoplastic Compound

Tumor embolization is a procedure used to reduce the vascularity of a tumor. A particulate form, such as microspheres, is administered via catheter, which is positioned in the tumor's arterial blood supply. The particles are released from the catheter and carried by blood flow to the arterioles and capillary bed where they embolize and thus retards blood flow to the tumor (Microspheres and Regional Cancer Therapy, CRC Press, Oct. 20, 1993). It is known that embolization induces a leakage of hepatic arginase from the liver into the circulation and the hepatic arginase released into the systemic circulation, in such a way, rapidly depletes plasma arginine (Cheng P N, Leung Y C, Lo W H, Tsui S M, Lam K C. Cancer Lett. Jun. 16, 2005;224(1):67-80. Epub Dec. 25, 2004.).

Thus, embolization effectively acts as a natural source of arginase that becomes released into the patient's blood stream after embolization i.e. it essentially acts as the administration of arginase to a patient, and is therefore expected to produce the same synergistic effect when administered in combination with an anti-neoplastic drug.

### EXAMPLE 9

### Using Dialysis as a Method to Reduce Physiological Arginine Levels in Combination with an Anti-Neoplastic Compound

Dialysis is a method used to remove waste materials and extra fluids from the blood. This method can also be used to remove certain amino acids from the blood (Gouyon J B, Desgres J, Mousson C. Pediatr Res. March 1994; 35(3):357-61). Therefore, dialysis can also be used to reduce the physiological levels of arginine in a human patient to below 10 μM, and is thus another implementation which may be used in combination with the administration of an anti-neoplastic drug.

While the present invention has been described using the aforementioned examples, it is clear that other combinations of drugs, may also have the same synergistic effect. An important aspect of the present invention is the recognition that arginine depletion is a new form of anti-neoplastic therapy that differs from the mode of operation of traditional anti-neoplastic drugs, such as, alkylating agents and mitotic inhibitors, etc.

Arginine deprivation results in the rapid and selective death of culture transformed and malignant cells (Scott L, Lamb J, Smith S, Wheatley D N. Br J Cancer. September 2000;83(6):800-10). It has been suggested that loss of viability in the malignant phenotypes may be the result of the loss of control primarily at the key G1 checkpoint, which normally prevents cells from reinitiating DNA synthesis under adverse conditions.

Therefore, whereas normal cells move into a quiescent state (G₀) during arginine deprivation, malignant cells which lack G1 checkpoint efficacy continue uncontrolled cell cycle advancement. The inventors of the present invention were able to recognize that uncontrolled cell cycle advancement of malignant cells under nutrient depletion in combination with cellular damage caused by an anti-neoplastic drug creates a significant synergistic cell killing.

In this description, nutrient depletion refers to the reduction of physiological arginine levels to below 10 μM. The reduction of physiological arginine levels may be achieved by different implementations. For this purpose, one implementation of the present invention is the administration of arginine reducing compounds to the human patient. Such arginine reducing compounds may be, but are not limited to, arginine degrading enzymes (such as arginase I, arginine decarboxylase, arginine deiminase, human, bovine and other animal arginase etc.). Another implementation of the present invention for this purpose is the use of embolization or dialysis, or other methods of treatment to reduce arginine levels.

Conventional anti-neoplastic drugs are directed to damaging different parts of the cell e.g. the metabolic pathways or DNA synthesis/repair/transcription. Families of anti-neoplastic drugs include, but are not limited to, alkylating agents such as chlorambucil, cyclophosphamide, thiotepa, and busulfan, mitotic inhibitors such as plant alkaloids (e.g. actinomycin D, mitomycin) and podophyllotoxins, and antibiotics (e.g. mitoxantrone, bleomycin), such as anthracyclines (e.g. doxorubicin). It is there part of the present invention that any one or combination of these drugs may be used as the cell-damaging agent referred to above.

In this description, 5FU is an example of an antimetabolite drug, and it is anticipated that other antimetabolite drugs, such as purine antagonists (e.g. 2-chlorodeoxyadenosine) and folate antagonists (e.g. methotrexate) would also provide synergistic effects.

In the most preferred embodiment of the present invention, 5FU is used in combination with administering arginase (BCT). 5FU is an analogue of uracil with a fluorine atom at the C-5 position in place of hydrogen. It rapidly enters the cell using the same facilitated transport mechanism as uracil. 5FU is converted intracellularly to several active metabolites, which disrupt RNA synthesis and the action of the nucleotide synthetic enzyme thymidylate synthase (TS) (Leffingwell R, Rustum Y. Fluoropyrimidines in Cancer Therapy. Humana Press. Jan. 1, 2003. p 61-62).

In Example 6 described above, the two compounds were administered at short time intervals, one immediately after another to facilitate experimental work. As used in this present invention, the term “combinatorial” or “used in combination” refer to the administration of the two compounds, such as an arginine degrading enzyme (e.g. arginase or arginine deiminase) and an anti-neoplastic compound such as 5FU, either simultaneously or consecutively within a temporal interval wherein the compound first administered is still at a concentration to exert an effect with regards to the treatment target cells, tissues or organs. The overlapping period of time in which the two compounds may be administered can be over a period of days or weeks, such as described in Example 7 above.

It is also not necessary that the routes of administration for the two compounds be the same. A compound such as 5FU may be administered topically. When used in human patients, the dosage of 5FU may be determined by a qualified health professional depending on the condition being treated, the size and overall health of the patient, as well as the particular regimen used. It is envisaged that when used in combination with an arginine degrading enzyme such as arginase (BCT) as taught by the present invention, the dosage of the anti-neoplastic compound such as 5FU can be significantly lower, thus also eliciting fewer and less severe side effects.

It must be noted that as used herein and in the appended claims, the singular forms “a,” “and,” and “the” include plural references unless the context clearly dictates otherwise. Thus, for example, reference to “a pharmaceutical preparation” includes mixtures of different preparations and reference to “the method of treatment” includes reference to equivalent steps and methods known to those skilled in the art, and so forth.

The invention having been fully described, modifications within its scope will be apparent to those of ordinary skill in the art. All such modifications are within the scope of the invention.

Formulations of the pharmaceutical composition of the present invention can be used in the form of a solid, a solution, an emulsion, a dispersion, a micelle, a liposome, and the like, wherein the resulting formulation contains one or more of the modified arginine degrading enzyme such as human arginase in the practice of the present invention, as active ingredients, in a mixture with an organic or inorganic carrier or excipient suitable for enteral or parenteral applications. The active ingredients may be the arginase, for example, with the usual non-toxic, pharmaceutically acceptable carriers for tablets, pellets, capsules, suppositories, solutions, emulsions, suspensions, and any other form suitable for use in manufacturing preparations, in solid, semisolid, or liquid form. In addition auxiliary, stabilizing, thickening and coloring agents and perfumes may be used. The active ingredients of one or more arginase are included in the pharmaceutical formulation in an amount sufficient to produce the desired effect upon the target process, condition or disease.

Pharmaceutical formulations containing the active ingredients contemplated herein may be in a form suitable for oral use, for example, as tablets, troches, lozenges, aqueous or oily suspensions, dispersible powders or granules, emulsions, hard or soft capsules, or syrups or elixirs. Formulations intended for oral use may be prepared according to any method known in the art for the manufacture of pharmaceutical formulations. The tablets may be uncoated or they may be coated by known techniques to delay disintegration and absorption in the gastrointestinal tract, thereby providing sustained action over a longer period. They may also be coated to form osmotic therapeutic tablets for controlled release.

In some cases, formulations for oral use may be in the form of hard gelatin capsules wherein the active ingredients are mixed with an inert solid diluent, for example, calcium carbonate, calcium phosphate, kaolin, or the like. They may also be in the form of soft gelatin capsules wherein the active ingredients are mixed with water or an oil medium, for example, peanut oil, liquid paraffin, or olive oil.

The pharmaceutical formulations may also be in the form of a sterile injectable solution or suspension. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,4-butanediol. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, or synthetic fatty vehicles, like ethyl oleate, or the like. Buffers, dextrose solutions preservatives, antioxidants, and the like, can be incorporated or used as solute to dissolve the soluble enzyme as required.

The pharmaceutical formulations may also be an adjunct treatment together with other chemotherapeutic agents.

In the claims, an arginase that has an amino acid sequence substantially the same as the sequence shown in SEQ ID No. 9 (amino acid sequence of normal human arginase I) means that the sequence is at least 30% identical to that shown in SEQ ID No. 9 or that using the Arginase activity assay as described herein, there is no significant difference in the enzymatic activity between the enzyme of SEQ ID No. 9 and the one that is substantially similar. The six histidines are provided for ease of purification, and the additional methionine group provided at the amino terminus thereof is to allow translation to be initiated. It is clear to one skilled in the art that other forms of purification may also be used, and therefore a “substantially similar” arginase does not need to have any homology with the MHHHHHH sequence of SEQ ID No. 3. In some bacterial strains there may be at least 40% homology with SEQ. SEQ ID No. 9. Some mammalian arginase may be 70% homology with SEQ ID No. 9.

SEQUENCE LISTING

| | |
|---|---|
| SEQ ID NO. 1: | (FIG. 2A) |
| SEQ ID NO. 3: | (FIG. 2B: amino acid sequence (SEQ ID NO:3) |
| SEQ ID NO. 5: | 5′-CCAAACCATATGAGCGCCAAGTCCAGAACCATA-3′ |
| | (Arginase I) |
| SEQ ID NO. 6: | 5′-CCAAACTCTAGAATCACATTTTTTGAATGACATGGACAC-3′ |
| | (Arginase II) |
| SEQ ID NO. 7: | 5′-CTCTGGCCATGCCAGGGTCCACCC-3′ (Arg 6) |
| SEQ ID NO. 8 & 9: | (FIG. 2C: nucleotide sequence (SEQ ID NO. 8); |
| | and amino acid sequence (SEQ ID NO. 9)) |

1. A method of treating cancer in a human patient, the method comprising reducing the physiological arginine levels in said patient to below 10 μM combined with administering an anti-neoplastic compound.

2. The method according to claim 1 wherein said method of reducing the physiological arginine levels in said patient comprises embolization, dialysis, or administering an arginine reducing compound.

3. The method according to claim 2 wherein said arginine reducing compound is an arginine degrading enzyme.

4. The method according to claim 3 wherein said arginine degrading enzyme is arginase, arginine deiminase, arginine decarboxylase, modifications thereof or combinations thereof.

5. The method according to claim 4 wherein said arginase is pegylated human arginase I.

6. The method according to claim 1 wherein said anti-neoplastic compound is an alkylating agent, antimetabolite agent, antimitotic agent, DNA production inhibiting agent or DNA repair inhibiting agent.

7. The method according to claim 1 wherein said anti-neoplastic compound is 5-fluorouracil.

8. The method according to claim 1 wherein said arginine reducing step is achieved by administration of pegylated human arginase I and 5-fluorouracil to a patient.

9. A kit for the treatment of human malignancies comprising at least one therapeutic dose of an arginine reducing compound and at least one therapeutic dose of an anti-neoplastic compound.

10. The kit according to claim 9 wherein said arginine reducing compound is an arginine degrading enzyme.

11. The kit according to claim 10 wherein said arginine degrading enzyme is arginase, arginine deiminase, arginine decarboxylase, modifications thereof or combinations thereof.

12. The kit according to claim 11 wherein said arginase is human arginase I.

13. The kit according to claim 11 wherein said arginase is pegylated human arginase.

14. The kit according to claim 9 wherein said anti-neoplastic compound is an alkylating agent, antimetabolite agent, antibiotic agent, DNA production inhibiting agent or DNA repair inhibiting agent.

15. The kit according to claim 9 wherein said anti-neoplastic compound is 5-fluorouracil.

16. A pharmaceutical composition comprising an arginine reducing compound and an anti-neoplastic compound.

17. The pharmaceutical composition according to claim 16 wherein said arginine reducing compound is an arginine degrading enzyme.

18. The pharmaceutical composition according to claim 17 wherein said arginine degrading enzyme is arginase, arginine deiminase, arginine decarboxylase, or modifications and combinations thereof.

19. The pharmaceutical composition according to claim 18 wherein said arginase is human arginase I.

20. The pharmaceutical composition according to claim 18 wherein said arginase is pegylated human arginase.

21. The pharmaceutical composition according to claim 16 wherein said anti-neoplastic compound is an alkylating agent, antimetabolite agent, antibiotic agent, DNA production inhibiting agent or DNA repair inhibiting agent.

22. The pharmaceutical composition according to claim 16 wherein said anti-neoplastic compound is 5-fluorouracil.

23. The pharmaceutical composition according to claim 16 wherein said pharmaceutical composition is a therapeutic dose for treating cancer

24. Use of an arginine reducing compound in combination with an anti-neoplastic compound for the manufacture of a medicament for the treatment of cancer.

25. The use according to claim 24, wherein said arginine reducing compound is 5 an arginine degrading enzyme.

26. The use according to claim 25, wherein said arginine degrading enzyme is arginase, arginine deiminase, arginine decarboxylase, or modifications and combinations thereof wherein said arginine reducing compound is an arginine degrading enzyme.

27. The use according to claim 26, wherein said arginase is human arginase I.

28. The use according to claim 26, wherein said arginase is pegylated human arginase.

29. The use according to claim 24, wherein said anti-neoplastic compound is an 15 alkylating agent, antimetabolite agent, antibiotic agent, DNA production inhibiting agent or DNA repair inhibiting agent.’

30. The use according to claim 24, wherein said anti-neoplastic compound is 5-fluorouracil.


SEQUENCE LISTING (5 sequences)

SEQ ID NO: 1 - DNA, 2002 bp, Homo sapiens
gaattgtacgtcaaagagatgaagcagaaaaacgtcgtcgagaagaagctgaacgacaaaaagtgaaatgcgagggaagtccaagaaatggtgattatgagggtgtctatttcaccaaaaacggagaatatttattggaattaagagtctctgggactgctcttgtaaatgctccttgtaatttaaaggatattgacataacgaaatggttgtgtaaaacagggagattatatcttgataaggttaagaaatttgaaatagttactattctttcccatgacgtagaaaatcaaaagattataacagaatgggagtcactccccagagaggctttacccgaacaatttgattcataagaactaattagtagcgctttccaatggaggcgcttttttatttgggtagttgcataccactaaagatgttcaggtgcacatgagcattggaggaaaggaacgctttagggggaagggaaacctttaaacagtcttaatcccccttgattttatgttctctgtaaactgcgtccggtaaatctcaggatagacaatcggcggttaacggcttgagtgcgggggcagtttagaaagaatatgattggagggattcatagatgcatcaccatcaccatcatatgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcacaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaaatgtggaaacatccgatataaatctcatagttaatggcataattagaaagctaatcattttcttaagcatagagttatccttctaaagacttgttctttcagaaaaatgtttttccaattagtataaactctacaaattccctcttggtgtaaaattcaagatgtggaaattctaacttttttgaaatttaaaagcttatattttctaacttggcaaaagacttatccttagaaagagaagtgtacattgatttccaattaaaaatttgctggcattaaaaataagcacacttacataagcccccatacatagagtgggactcttggaatcaggagacaaagctaccacatgtggaaaggtactatgtgtccatgtcattcaaaaaatgtgattctaga

SEQ ID NO: 2 - DNA, 990 bp, Homo sapiens
atgcatcaccatcaccatcatatgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcacaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaa

SEQ ID NO: 3 - protein, 329 aa, Homo sapiens
MHHHHHHMSAKSRTIGIIGAPFSKGQPRGGVEEGPTVLRKAGLLEKLKEQECDVKDYGDLPFADIPNDSPFQIVKNPRSVGKASEQLAGKVAQVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 4 - DNA, 969 bp, Homo sapiens
atgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcacaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaa

SEQ ID NO: 5 - protein, 322 aa, Homo sapiens [sequence omitted]